(19) United States
(12) Patent Application Publication
(10) Pub. No.: US 2021/0346554 A1
(43) Pub. Date: Nov. 11, 2021

(54) PERSONAL PROTECTIVE EQUIPMENT

(71) Applicant: Kambiz Behzadi, Pleasanton, CA (US)

(72) Inventors: Kambiz Behzadi, Pleasanton, CA (US); Michael E. Woods, Brisbane, CA (US)

(73) Assignee: Kambiz Behzadi, Pleasanton, CA (US)

(21) Appl. No.: 16/993,066

(22) Filed: Aug. 13, 2020

Related U.S. Application Data
(60) Provisional application No. 63/022,524, filed on May 10, 2020.

(51) Int. Cl. A61L 2/24; A61L 2/04; A61L 2/10; A61L 2/08; A61L 2/14; A61L 2/025; A61L 2/20; A61L 2/22; A61L 9/16; A61L 9/20; A61L 9/18; A61L 9/22; A61L 9/04; A61L 9/03; A61L 9/14; A41D 31/30

(52) U.S. Cl. CPC A61L 2/24; A61L 2101/02; A61L 2/10; A61L 2/084; A61L 2/14; A61L 2/025; A61L 2/202; A61L 2/22; A61L 9/16; A61L 9/20; A61L 9/18; A61L 9/22; A61L 9/046; A61L 9/03; A61L 9/14; A41D 31/30; A61L 2/04

(57) ABSTRACT

A system and method for adoption and use of social personal protective equipment. A structure, such as an article of clothing or jewelry, incorporates one or more PPE features and produces a protective, non-physical barrier around the user that kills or degrades a targeted, or group of, pathogenic microorganisms.

### CROSS REFERENCE TO RELATED APPLICATIONS

This Application claims the benefit of U.S. Provisional Application 63/022,524 filed on May 10, 2020, the contents of which are hereby expressly incorporated by reference thereto in its entirety for all purposes.

### FIELD OF THE INVENTION

The present invention relates generally to personal protective equipment, and more specifically, but not exclusively, to non-physical personal barrier equipment associated with a person to minimize exposure to communicable hazards that may cause social illnesses. These illnesses may result from contact with physical (e.g., pathogenic microorganisms) environmental hazards. The non-physical barrier may be used in addition to, or in lieu of, personal physical barriers, and non-physical non-personal barriers.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

People worldwide have been dramatically affected by the emergence of the novel coronavirus pandemic, which has resulted in severe illness and caused untold number of deaths.

Current attempts to protect against catching the virus include (i) personal protective equipment (PPE) which include gloves, masks, gowns, and face shields and (ii) a variety of sanitizers including alcohol, detergents, and many different kinds of chemicals.

The PPE are worn as a physical hard barrier to keep the virus from entering the body, however, the current concept of PPE has severe limitations. Some of these limitations arise from use of specialized PPE by persons unfamiliar with appropriate procedures for donning, using, and removing the equipment as well as widespread use which increases risks associated with improper use.

The manner in which such PPE is applied and removed involves multiple steps where simple and common errors can cause contamination. For example, removing one's gloves before removing one's gown, or touching one's face to remove one's mask with contaminated gloves, are a few of many examples, in which every step of the current PPE donning on/off process is prone to contamination.

Therefore, PPEs as currently deployed are highly unreliable, simply because they themselves get contaminated (are “contaminable”) with a virus/bacterium (pathogenic microorganism), and then the PPE can secondarily contaminate the user and/or the user's environment.

A second limitation of the current art is that the PPE do not themselves have a capacity to sanitize or kill pathogens. Humans have to remove the PPE with proper and perfect form and then use chemicals or alcohol to sanitize their hands. Some PPE may be subject to sanitization and re-use provided appropriate procedures and precautions are used. Therefore, current PPEs in use are not (i) “uncontaminable” and (ii) do not sanitize or kill pathogens.

A third limitation of the current PPE system is that they wait too long to protect humans because they are worn ON the body: gloves, mask, gowns and shields. The PPE are attached to the body from a range of range of 0.1 cm (glove) to 10 cm (shield), and the pathogenic microorganisms are resident on the PPE.

This concept of PPE is essentially borrowed from healthcare workers in the hospital, ER, OR and doctors' offices. However, this system of PPE may not be the best option (suitable) for the general public who want to interact with each other in the social setting. Potential intimate partners may not find a user of such conventional PPE appealing when donning a gown, mask/shield, and gloves.

Another potential limitation of the PPE is that they really do not protect humans from aerosolized particles in the air that carry the virus. Wearing a mask is not going to protect your hair, eyes, face and arms from a highly attachable pathogenic microorganism, 20 million of which may be aerosolized in a sneeze or a cough.

An additional limitation of the current PPE system is that they are physical hard structures which essentially make it easy for attachment of the pathogenic microorganisms and carriers of such pathogenic microorganisms (mucous, saliva, dander, and the like).

Conventional personal physical barrier equipment are a subcategory of what are referred to as fomites. A fomite is an inanimate object that, when contaminated with or exposed to infectious agents, can transfer disease to a new host. The role of fomites in disease transfer is high because of increased gatherings of groups of people indoors.

Adoption of PPE by in social situations may be facilitated by improving on the conventional PPE architecture, particularly when such social-friendly PPE could be incorporated in different form factors that may include or convey a non-medical/non-physical barrier “vibe” and may actually enhance the social adoption of PPE.

### BRIEF SUMMARY OF THE INVENTION

Disclosed is a system and method for adoption and use of social personal protective equipment. The following summary of the invention is provided to facilitate an understanding of some of the technical features related to non-physical barrier personal protective equipment, and is not intended to be a full description of the present invention. A full appreciation of the various aspects of the invention can be gained by taking the entire specification, claims, drawings, and abstract as a whole. The present invention is applicable to other potential environmental hazards in addition to pathogenic microorganisms, and in some cases, may be applicable to protect air flow corridors between volumes frequented by persons who may carry or shed environmental hazards (e.g., HVAC ducting in nursing homes, prisons, and the like).

Attributes of an improved social PPE may include one or more of: (i) uncontaminable (cannot be contaminated, or resists contamination, and therefore avoids/resists/decreases secondarily contamination); (ii) protection arises because it degrades, sanitizes or kills pathogens; (iii) not a physical structure that can be easily attached to by the microorganism or carrier (non-attachable); (iv) behaves like a protective shield as in the nonphysical “deflector shield” of the USS Enterprise protecting against an enemy's torpedoes; (v) behaves like ray gun (to pathogens only) as in USS Enterprise “phasers”; and/or (vi) does not conceal the identity of a person wearing the PPE including revealing facial features that are important for security and quality social interaction.

An embodiment of the present invention may include a wearable or associable structure, for example an article of clothing or jewelry, that provides one or more PPE features for the user.

A question is why wait until an enemy (a pathogenic microorganism) is inside your castle (body) to deploy countermeasures? One would prefer that the security would include lethal weapons but a range of options are in fact reduced once the microorganism is inside the perimeter. These limitations are intended to reduce collateral damage of the host. Stronger security measures may be employed before the pathogen has entered into the body and body components (cells).

Your defense systems should never provide a major advantage for the enemy. Physical PPEs are physical structures which by definition betray the goal of protection because many pathogenic microorganisms thrive on physical structures. Structureless PPEs allow removal or reimagining of traditional PPE.

An embodiment may include a personally associable system that moves with the user (e.g., worn) to create a non-physical protection zone around the user, such as the face and hands. The non-physical protection zone may include a combination of microbicidal agents that define parameters for this non-physical protection zone and have been provided into the user's personal space by the system.

An embodiment may include a set of systems distributed to a plurality of individuals who have gathered together such as in a closed space. When each person of the gathering uses a personally associable system, an “undistancing” synergy occurs as these individual non-physical protection zones may constructively reinforce each other and produce a group non-physical protection zone. An effectiveness of this group non-physical protection zone may actually increase as the separation distance between individuals decreases. In some situations, the group non-physical protection zone may offer suitable protection for a few members of the gathering to dispense with use of a personally associable system. This reinforcing synergy of the collective overlapping individual protection zones may be thought of as a type of “herd protection” for the individuals of the gathering.

An embodiment may include protective field generators that shape and contour the individual protective zone based upon local environmental conditions. This environment includes an assessment of local risk profiles and possible attack vectors of pathogenic microorganisms nearby the user. The system may change the parameters of the agents used in the personal protective zone based upon the profiles/vectors to reduce risks/mitigate attack vectors. For example, a gathering with few people spaced apart may operate the system with a reduced intensity for and/or agent-type selection of the agents within the protective zone. As the number of people increases and/or the average distances around the user decreases, the system may increase the intensity and/or change the agent-type appropriately.

An embodiment may include communication modalities allowing each individual to receive third-party information for configuring each individual's protective zone. A facility where a gathering occurs may have minimum necessary thresholds for the protective zone parameters for users within the facility. A facility operator may compile and distribute occupancy and pathogen risk data that may be used by individual systems to tune the individual protective zone appropriately.

A system for a user, the system providing individual protection against a pathogenic microorganism, including a set of components configured for association with the user, the set of components adapted to move with the user during operation, the set of components including a source of power, a controller, coupled to the source of power, executing a set of instructions retrieved from a memory to produce a set of control signals, and a set of microbicidal generators, coupled to the source of power and responsive to the set of control signals, providing a structureless microbicidal agency projected within a protective zone proximate a set of anatomical features of the user, wherein the microbicidal agency destroys, destroys, disturbs, denatures, or otherwise reduces a pathogenicity or transmission of a pathogenic microorganism within the protective zone before the pathogenic microorganism contacts one the anatomical feature of the set of anatomical features.

A wearable device (personal protective equipment) for protecting humans against transmission of surface, airborne and aerosolized pathogens (virus, bacteria and fungus, and the like) comprising a non-physical, invisible, deflector shield (Mandorla=“personalized protective electromagnetic/chemical field” PPE/CF—capable of non-physical barrier pathogen control), produced from a wearable device including (i) power supply; (ii) natural and/or artificial light, electromagnetic, radioactive and chemical elements; (iii) miniaturized electronics, microchips and microcontrollers; and (iv) optionally a wireless communications system; and optionally the wearable device includes a non-physical electromagnetic/chemical field is created around the body; and optionally the wearable device wherein pathogens in the vicinity of the body are degraded or destroyed at a distance away from the body before contacting and/or entering the body; and optionally wherein the wearable device includes the pathogen destroying element includes a natural light or electromagnetic, or radioactive elements; synthetic (artificial) chemical elements, a combination of natural and artificial elements; and optionally wherein the wearable device may be installed in one or more wearable structures selected from the group consisting of jewelry, hats, gloves, clothing, masks, shields, and gowns, and combinations thereof.

A method of destroying (killing/degrading viability) pathogens (virus, bacteria, fungus) with a “personalized protective electromagnetic chemical field” (PPE/CF) prior to entrance of pathogen into the body with stronger lethal chemical and/or electromagnetic phenomena as opposed to weaker oral, subcutaneous, intravenous and intramuscularly delivered drugs (antibiotics) commonly used to treat pathogens during infectious process after infection/invasion. The method where the PPE/CF creates a 0.1 meter to 2-meter diameter virtual bubble like field around the wearable, and optionally wherein the PPE/DF is damaging, poisonous or harmful to pathogens but innocuous, benign and harmless to humans; the method wherein a non-structural, non-physical-barrier disinfection field decreases a probability of human contamination, by decreasing a total surface area available for virus and bacteria to land on, through elimination/reduction of cPPE; the method wherein killing pathogens in close vicinity of human body occurs before gaining entrance into human body; the method wherein humans are protected from airborne and aerosolized pathogens without having to wear a full complement of physical structured personal protective barrier equipment including gowns, spacesuit, purified air system, masks, shields and the like; the method wherein the PPE/CF is comprised of a “personal Far UVC field” PUVCF, comprising of far UVC light in the vicinity of (207 nm to 222 nm), where the light cannot penetrate outer layers of human skin or eye, but can efficiently kill airborne aerosolized viruses such as influenza, H1N1, SARS-COV-2 and other corona virus as well as other pathogens, called PUVCF; the method wherein UVC light is not exclusively shone from the ceiling or a sidewall but includes UVC light produced from the portable/mobile wearables associated with a human body; the method wherein a distance traveled by far UVC light is less than 7 to 8 feet; the method wherein a direction of far UVC light is away from an associated human body rather than towards the associated human body, as typically occurs from an exclusively ceiling, wall or flood light stand system; the method wherein a source of origin of the far UVC light is within 6 to 12 inches of the human body; the method wherein a source of the far UVC light moves and travels with the human body (and is not stationary on a wall, ceiling or stand); a method increasing and enhancing an effectiveness of PPE/CF by congregating a multiplicity of PPE/CFs around the same location, where multiple PPE/CFs operate around the human body as germicidal nodes or germicidal beacons individually, but with cooperation together, constitute a distributed disinfection field (DDF), where the global aggregate operating system that is produced is exponentially stronger and more effective in killing pathogens than the single PPE/CF. This is called the “densification process”; the method wherein the DDF includes several autonomous nodes/beacons independently producing viricidal properties, where the nodes/beacons communicate with each other to solve a common problem, to decrease a count of viable pathogens in air and on surfaces surrounding a group of humans; the method wherein (a) the system tolerates failure in individual beacons; (b) where the structure of the system may change during the execution of the distributed function (e.g., increase or decrease its effectiveness based on participation of nodes, (c) where disinfection is distributed across a cluster, grid or cloud and maybe enhanced as the number of beacons nodes are increased; and a method of creating surgical gowns with multiple germicidal nodes within the gown to create a distributive cooperative nodes of PUVCF around the surgeon's body “multi-noded PPE/CF, which can produce a personalized DDF around the surgeon; and wherein the method wherein multiple staff, surgeons, and other participants in a procedure wear personalized DDFs configured to create a “super distributed disinfection field” SDDF in the OR space to significantly decreases the incidence of surgical site infections SSI.

Any of the embodiments described herein may be used alone or together with one another in any combination. Inventions encompassed within this specification may also include embodiments that are only partially mentioned or alluded to or are not mentioned or alluded to at all in this brief summary or in the abstract. Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

Other features, benefits, and advantages of the present invention will be apparent upon a review of the present disclosure, including the specification, drawings, and claims.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1-FIG. 11 illustrate various embodiments for improved environmental hazard reduction systems such as may be incorporated into personal protective equipment limiting exposure to pathogenic microorganisms that may be present in a user's vicinity;

FIG. 1 illustrates an embodiment for eyewear that incorporates a PPE feature;

FIG. 2 illustrates an embodiment for jewelry that incorporates a PPE feature;

FIG. 3 illustrates an embodiment for a watch that incorporates a PPE feature;

FIG. 4 illustrates a set of clothing articles that incorporate a PPE feature;

FIG. 5 illustrates an article of hand wear that incorporates a PPE feature;

FIG. 6 illustrates an article of head wear that incorporates a PPE feature;

FIG. 7 illustrates an article of clothing incorporating an alternative PPE feature;

FIG. 8 illustrates a set of cooperative components including an aerosol PPE feature;

FIG. 9 illustrates an article of hand wear that incorporates an alternative PPE feature;

FIG. 10 illustrates an article of head wear that incorporates an alternative PPE feature;

FIG. 11 illustrates an article of head wear that incorporates another alternative PPE feature;

FIG. 12 illustrates a conventional ceiling-mounted UV distribution system;

FIG. 13 illustrates a conventional wall-mounted UV distribution system

FIG. 14 illustrates a first hat with an architecture providing PUVCF;

FIG. 15 illustrates a second hat with an architecture providing PUVCF;

FIG. 16 illustrates a third hat with an architecture providing PUVCF;

FIG. 17 illustrates a fourth hat with an architecture providing PUVCF;

FIG. 18 illustrates a representation of a person equipped with a personal wearable protective-zone generator;

FIG. 19 illustrates a first gathering of a first set of persons, each person equipped with a personal wearable protective zone generator, the set collectively producing a distributed reinforcing group protective zone;

FIG. 20 illustrates a second gathering of a second set of persons depicting an increased level of protective intensity, directly related to the number and proximity for the distributed reinforcing group protective zone illustrated in FIG. 21; and

FIG. 21 illustrates a personal protective equipment including a combination of conventional technology and field emitters as described herein to improve protection for first responders and health care professionals frequently in close proximity to third-parties who are infected or whose infection status is unknown.

### DETAILED DESCRIPTION OF THE INVENTION

Embodiments of the present invention provide a system and method for adoption and use of social personal protective equipment. The following description is presented to enable one of ordinary skill in the art to make and use the invention and is provided in the context of a patent application and its requirements.

Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

### Definitions

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The following definitions apply to some of the aspects described with respect to some embodiments of the invention. These definitions may likewise be expanded upon herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

As used herein, the singular terms “a,” “an,” and “the” include plural referents unless the context clearly dictates otherwise. Thus, for example, reference to an object can include multiple objects unless the context clearly dictates otherwise.

Also, as used in the description herein and throughout the claims that follow, the meaning of “in” includes “in” and “on” unless the context clearly dictates otherwise. It will be understood that when an element is referred to as being “on” another element, it can be directly on the other element or intervening elements may be present therebetween. In contrast, when an element is referred to as being “directly on” another element, there are no intervening elements present.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common properties.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “connect,” “connected,” and “connecting” refer to a direct attachment or link. Connected objects have no or no substantial intermediary object or set of objects, as the context indicates.

As used herein, the terms “couple,” “coupled,” and “coupling” refer to an operational connection or linking. Coupled objects can be directly connected to one another or can be indirectly connected to one another, such as via an intermediary set of objects.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable properties that are substantially the same as those of the non-spherical object. Thus, for example, a size of a non-spherical object can refer to a diameter of a corresponding spherical object that exhibits light scattering or other properties that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

As used herein, the term “conventional personal protective equipment” (PPE or cPPE as context indicates) means protective clothing, helmets, gloves, face shields, goggles, facemasks and/or respirators or other equipment designed to protect the wearer from injury or the spread of infection or illness. CPPE is commonly used in health care settings such as hospitals, doctor's offices, and clinical labs. When used properly, CPPE may act as a physical structural barrier between infectious materials, including pathogenic microorganisms such as viral and bacterial contaminants, and the wearer's select anatomical features, including skin, mouth, nose, or eyes (e.g., mucous membranes). The physical structural barrier has the potential to physically block/obstruct transmission of contaminants to the wearer from environmental blood, body fluids, or respiratory secretions.

As used herein, the term “structureless personal protective equipment” (PPE or sPPE as context indicates) means protective components designed to generate non-structural-barrier protection to the user from injury or the spread of infection or illness. SPPE act as a non-physical microbicidal zone between infectious materials, including pathogenic microorganisms such as viral and bacterial contaminants, and the wearer's select anatomical features, including skin, mouth, nose, or eyes (e.g., mucous membranes). The non-physical microbicidal zone has the potential to non- physically interfere, disrupt, or otherwise non-physically stop or inhibit transmission of contaminants to the wearer from environmental blood, body fluids, or respiratory secretions.

As used herein, the term “microorganism” or “microbe” means a microscopic organism, including those of medical interest such as bacteria, fungi, archaea, and protozoa, with a particular focus on pathogenic organisms. Viruses and viroids are herein also included as microorganisms, it being recognized they are sometimes excluded because they are not cellular and they are unable to replicate without a host cell. A microbiocidal agency, agent, or element destroys, degrades, disturbs, denatures, deactivates, disrupts or interferes with one or more such microorganisms to reduce or eliminate pathogenic activity of the microorganism or reduce or eliminate transmission or communication of the pathogenic microorganism to reduce pathogenicity.

As used herein, the term “personal protective zone” means an intangible region or mandorla formed around, and associated with, a user. This personal protective zone moves with the user and may have a variety of microbicidal agents or agencies that degrade/destroy/deactivate/disturb/denature pathogenic microorganisms, or transmission thereof, coming into a vicinity of the user. The personal protective zone originates from nearby the user and extends outward away from the user towards a source or reservoir of pathogenic microorganisms. The character, shape, composition, and/or operational parameters of the region/mandorla may be variable based upon a range of characteristics and design goals as further described herein.

As used herein, the term “protective field projector” means a structure or system associated with a user's body that moves/follows the user during social interaction activities, the structure or system providing a personal protective zone for the user during operation. In some cases, the protective field projector may be fixed as to the characteristics of the personal protective zone provided the user. In other cases, the protective field generator may have a facility for altering or adjusting one or more characteristics of the personal protective zone provided the user. The alteration/adjustment may be manually set by a user, it may be responsive to configuration information provided by a third party, or a combination thereof.

As used herein, the term “distributed protective zone” means an intangible region or mandorla formed around, and associated with, a group, collection, or gathering of users within a spatial volume. This distributed protective zone is a collateral response to reinforcing overlapping personal distributed zones associated with each user of a subset of the group of users. This subset of users is close enough to one another so that the personal protective zones begin to overlap and provide a secondary synergistic field to further protect the individual users by protecting the group of users. The distributed protective zone may respond to the aggregate motions and zone characteristics of the users of the group and have a variety of microbicidal agents or agencies that degrade/destroy pathogenic microorganisms coming into a vicinity of the user. With an ad hoc distributed protective zone, the character, shape, composition, and/or operational parameters of the distributed protective zone may be variable based upon a range of characteristics and design goals of the contributing personal protective zones and protective field generators of the users of the group as further described herein. By having a possibility of a wider range of microbicidal agents or agencies participating in the distributed protective zone than may be possible from any single user, the distributed protective zone may provide a superior level of protection for group members than if those people were alone.

There is a large amount of knowledge and existing science regarding the phenomena, many of which are natural, of killing and retarding bacterial, viral, and fungal pathogens (sometimes referred to herein as pathogenic microorganisms), which include electromagnetic, physical, radioactive, and chemical processes and agencies.

These include, without limitation: (i) heat (especially greater than 56C), and the process of drying/desiccation; (ii) UV light (especially the Far-UVC light , 207-222 nm) (UV—100 to 400 nm); (iii) Ozone O₃; (iv) ions, as in Zinc, Iron, and Copper ions (+/− mixed with peroxide); (v) visible light and its phototoxic affects (400 nm to 800 nm) of intense pulses of visible light; (vi) humidity; (vii) ionizing or non-ionizing radiation, electron beams, gamma rays, x-rays with appropriate energy for sufficient degradation of the targeted pathogenic microorganism(s) while having an energy level safe for humans nearby the operating system; (viii) aerosolization of chemicals (fogging); (ix) pulsed lasers tuned to a degrading/destructive resonance frequency of a class or specific pathogenic microorganisms, (x) ultrasonic energy which may also be tuned, and (xi) air pressure phenomena (negative and positive air pressures); and there is also a concept of a microbicidal agent that kills microorganisms such as bacteria, while a microbiostatic agent only prohibits the growth of such microorganisms;—collectively sometimes referred to herein as a PPE, or sPPE, feature.

With respect to electromagnetic energy, including heat, UV, and visible light, these are emitted by the sun and received by earth and its inhabitants. Humans receive this energy (receiver). Sun emits energy (emitter).

It is an aspect of some embodiments of the present invention that any combination of these sources may be combined/integrated together and applied to wearables/associables on human body to essentially make the human body “a sun like electromagnetic emitting structure”, essentially producing a human with a “deflector shield”.

For example, the proper dosages of UV light, intense pulses of visible light, and heat can be determined to provide maximum protection for the human body by killing pathogens without harming humans.

Electromagnetic energy such UV light can be mixed with chemical energy including, for example (Zn+ ions) and O₃ in just the right dosages (likely minimal amounts) to be highly effective in killing/degrading bacteria and viruses.

These sources of energy and sPPE features may be attached to, or associated with, the human body through wearables, including hats, caps, eyewear, sunglasses, watches, earrings, necklaces, rings, gloves, gowns, face shields, and clothing (shirts) to produce a combination of properly dosed killing/degrading, non-physical “phaser rays” and “deflective shields”.

The rays will provide a trajectory to any object the human (eyes or hand) point or looks at, such as looking to a direction or pointing to a direction. The rays may be limited in the total distance or area they cover, such as to humans' personal space (maybe 18 inches or 2 ft)

The shield will provide a deflector bubble around the human anywhere from 5 cm to 100 cm away from the human body, as in the form of an umbrella or mushroom.

It is noteworthy that many of these existing technologies are highly viricidal and bactericidal in and of themselves, at lower doses that are non-harmful humans. Furthermore, it is especially conceivable that several of these technologies can be packaged in conjunction (as a mixture) with each other to derive an especially potent (not yet discovered) killer of bacteria and viruses, to be deployed as a shield or ray gun, through a wearable, around the human body, while maintaining excellent safety profile for humans.

Further, some embodiments of the present invention provide a synergistic effect by combining overlapping protection “fields” from other users in the nearby area. One of the challenges is that risks of pathogenic communication from one person to another increases as the number of people in an area increases and as the average separation distance decreases. Some of the sPPE features may be implemented so that the effectiveness increases linearly or exponentially as the number of people increases and as the average separation distance decreases. This can be considered as extra protection with “social undistancing” or “enhanced herd protection” which is counterintuitive to current thinking and technology.

An example may include far-UVC light combined with Zn ionizer and heat ray. Another example maybe low dose O₃ combined with intense pulsed of visible light and humidity.

Additionally, currently certain technologies are used to kill pathogens in meat, poultry and produce. These include radiant energy including gamma rays, electron beams and x-rays. It is conceivable that these technologies can be miniaturized and utilized at even much smaller doses. They can then be emitted from wearables to produce virus killing sPPE.

Similarly, currently chemicals such as hydrogen peroxide, benzalkonium, chlorohexidine, betadine are used to kill bacteria on surfaces. It may be possible that these chemicals can be aerosolized in humidifier or fogging systems and deployed on wearables at extremely low doses such they produce no harm to the humans but kill virus and bacteria on contact.

Finally, physical phenomena such as negative and positive pressure environments can be created in wearables (shirts, hats, necklaces and the like.) in order keep contagious and harmful bacteria away from a person. For example, miniaturized engines with ventilation system embedded in a shirt can constantly produce clean air moving away from the host producing a pressure phenomenon around the host that physically keeps the virus away by blocking aerosolized viral pathogens. There are also solid state “fans” or other air movement systems that operate without moving fan blades, such as electrostatic systems that generate air flow. In some instances, it is possible to redesign clothing elements to enhance sPPE features—such as providing high and stiff collars that create air channels or pathways that concentrate and direct air movement in desired locations (e.g., moving air away from the user's face or hands) to improve the associated sPPE feature.

Ultimately, any combination of the above technologies at proper and adjusted doses can be admixed together, the systems and/or system effects, to produce an improved sPPE.

There is tremendous room for experimentation to determine the best combination of above electromagnetic, physical and chemical technologies to kill viruses and bacteria, and to incorporate this technology into wearables, in order to produce “shields” and “rays” that not only kill viruses but do not provide a physical structure to which the virus can attach.

These various forms of radiant energies, ions, aerosols and air pressures will be produced through battery operated, chargeable miniaturized machines, integrated circuits, microchips, and microcontrollers (minicomputers); and attached through various means (adhesives and stitching) to wearables.

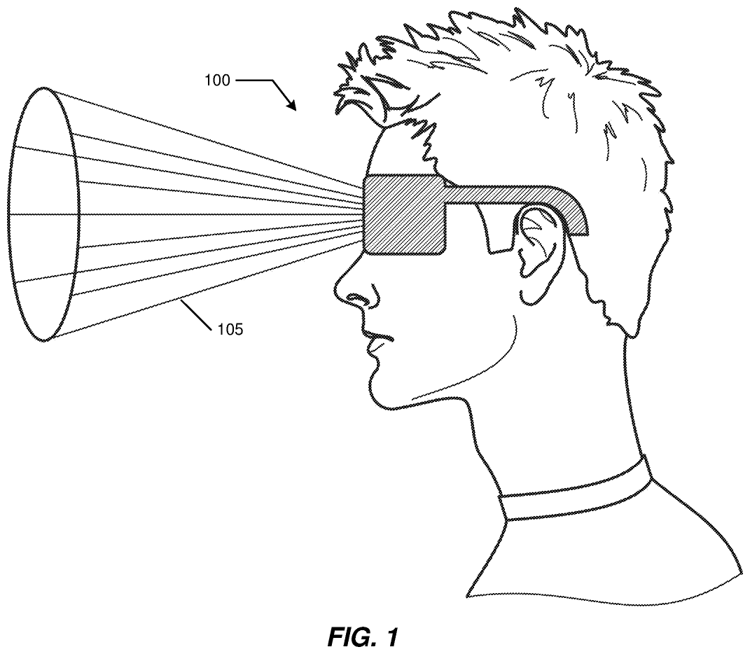

FIG. 1-FIG. 11 illustrate various embodiments for improved environmental hazard reduction systems such as may be incorporated into personal protective equipment limiting exposure to pathogenic microorganisms that may be present in a user's vicinity;

FIG. 1 illustrates an embodiment for eyewear 100 that incorporates an sPPE feature 105. Eyewear 100 presents the sPPE feature in a way that pathogenic organisms present in the field-of-view of the user (e.g., surfaces such as a tabletop or a nearby window) are killed or sufficiently degraded.

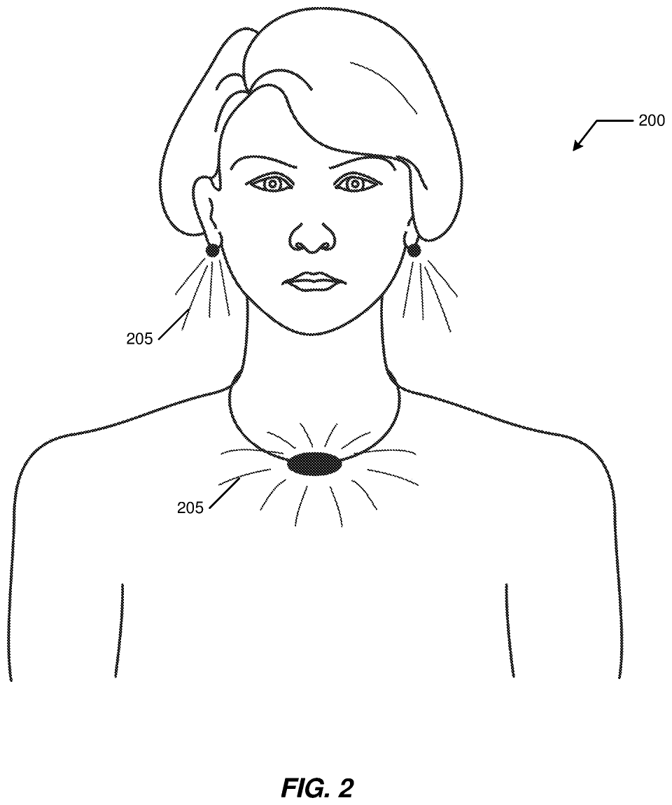

FIG. 2 illustrates an embodiment for jewelry 200 that incorporates an sPPE feature 205. As illustrated, a necklace and earrings may incorporate a UV/ozone sPPE feature to generate and maintain a protective shield around the face and neck, the protective shield killing or degrading targeted pathogenic microorganisms.

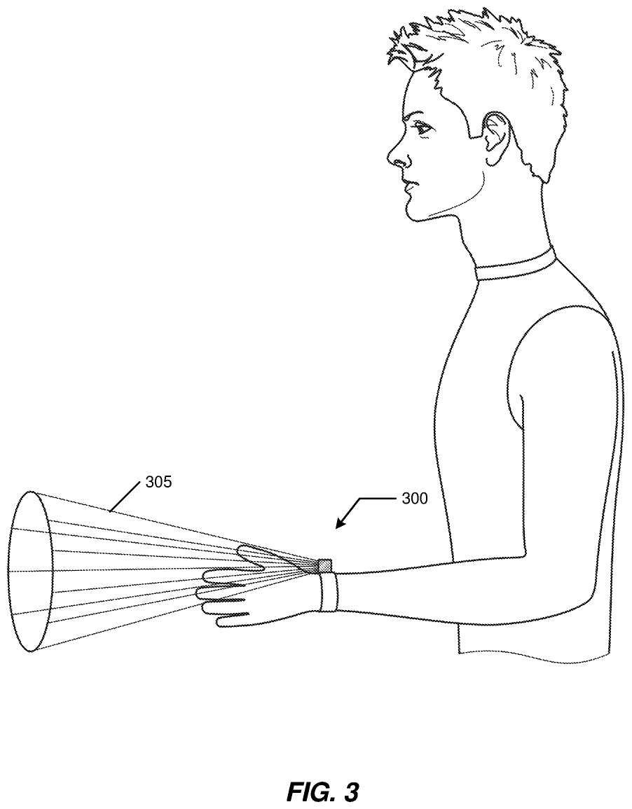

FIG. 3 illustrates an embodiment for a watch 300 that incorporates an sPPE feature 305. As illustrated watch 300 or bracelet may incorporate sPPE feature 305 that creates overlapping columns UV rays that kill or degrade target pathogenic microorganisms before being touched by the user.

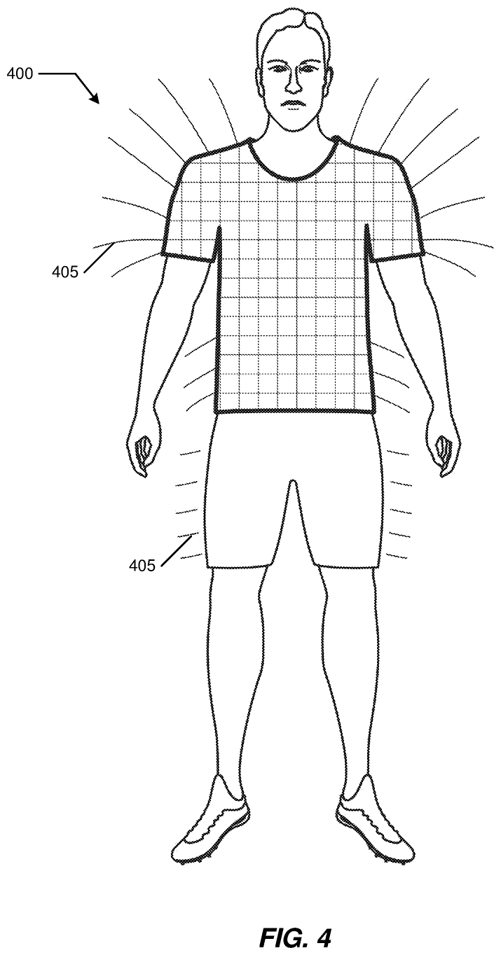

FIG. 4 illustrates a set of clothing articles 400 that incorporate an sPPE feature 405. As illustrated, one or both of a shirt 400 and pants 400 incorporate sPPE feature 405 that emits a field of UV energy around the user to kill or degrade pathogenic microorganisms.

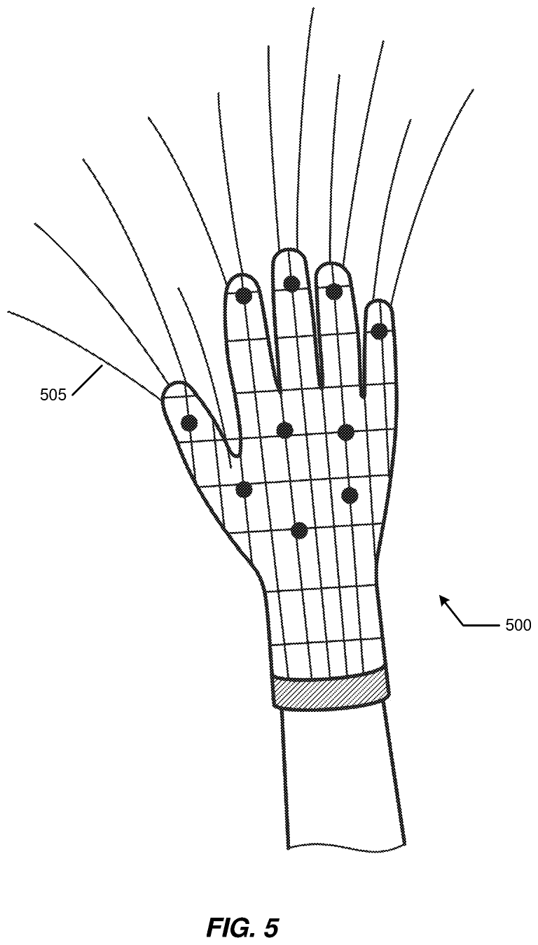

FIG. 5 illustrates an article of hand wear 500 that incorporates an sPPE feature 505. As illustrated, glove 500 or mitten 500 incorporate sPPE feature 505 that emits a field of UV energy. Glove/mitten 500 may include tessellated/segmented electronics for the generation of this UV energy field.

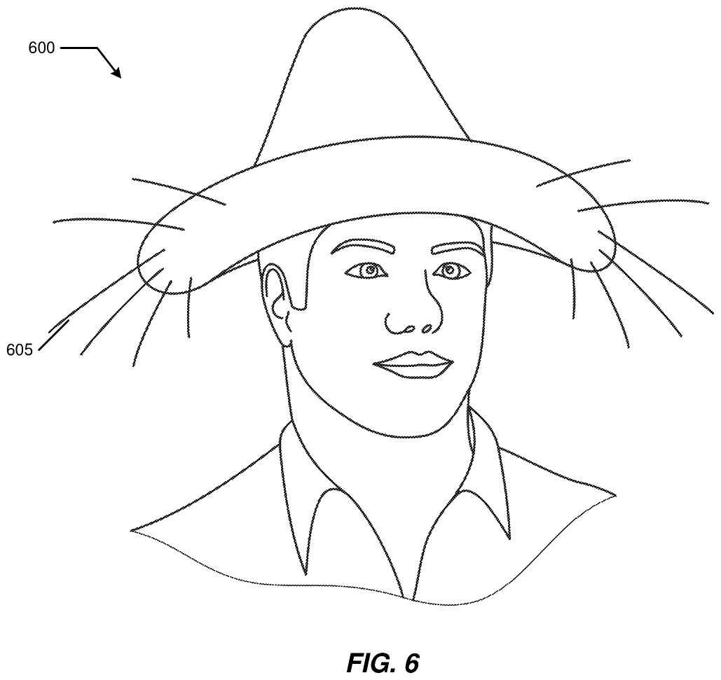

FIG. 6 illustrates an article of head wear 600 that incorporates an sPPE feature 605. As illustrated, an air motion system generates an air pressure differential that moves air away from the user's head. In some systems, an aerosol may be introduced into this air pressure differential to kill or degrade the targeted pathogenic microorganism.

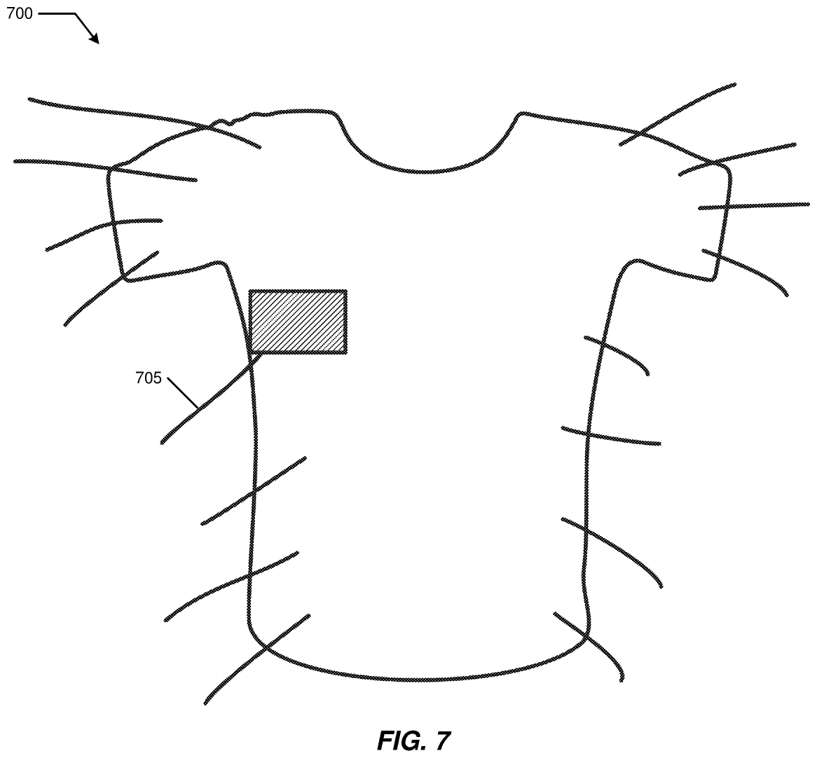

FIG. 7 illustrates an article of clothing 700 incorporating an alternative sPPE feature 705. Similar to FIG. 6, an air motion system may be incorporated into a shirt or vest and move air that may contain the environmental hazard away from the user.

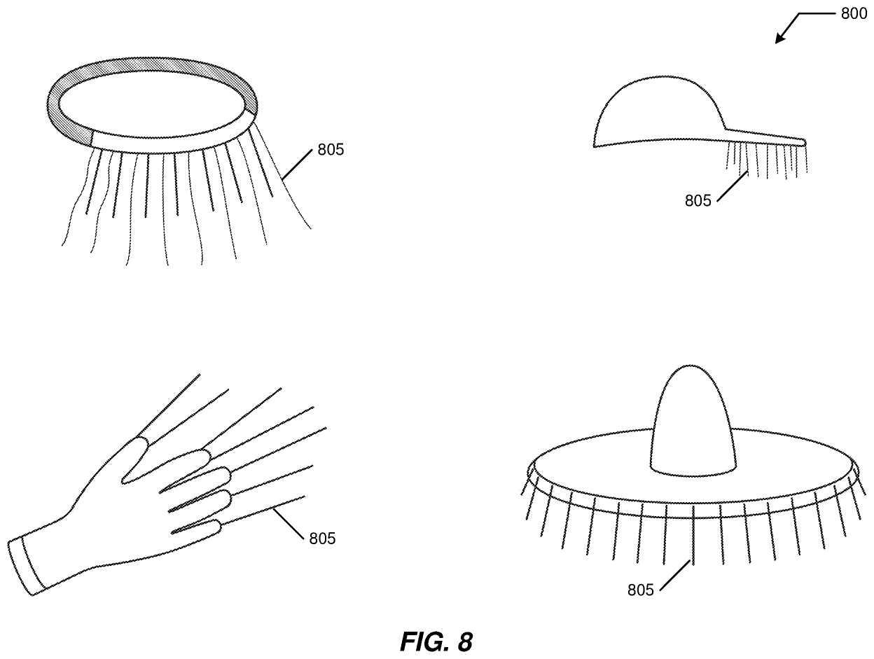

FIG. 8 illustrates a set of cooperative components 800 including an aerosol sPPE feature 805. Set of cooperative elements 800 may include a glove, a headband, a hat, or cap, each component including an aerosol producing system to generate a protective field sPPE feature 805 around the user for killing/degradation of a targeted pathogenic microorganism.

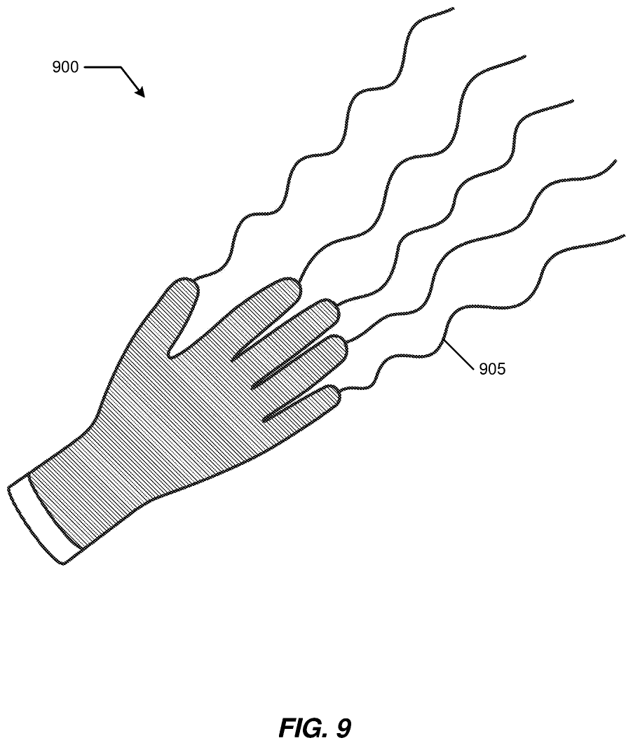

FIG. 9 illustrates an article of hand wear 900 that incorporates an alternative sPPE feature 905. As illustrated, a glove or mitten is configured to generate radiation that kills or degrades a targeted pathogenic microorganism. The radiation may be of an energy or character for ionizing the microorganism without harming the user or other nearby people or animals—a safe dose for the user may be destructive to the targeted pathogen.

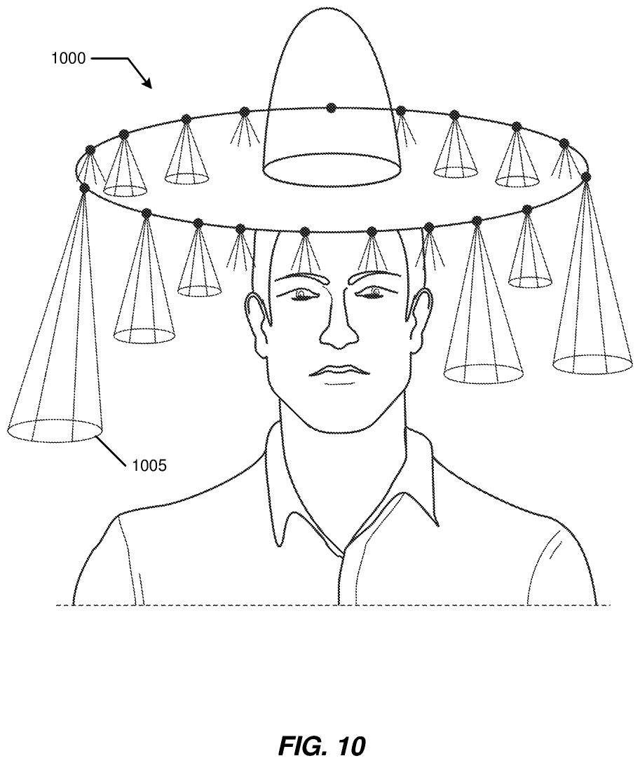

FIG. 10 illustrates an article of head wear 1000 that incorporates an alternative sPPE feature 1005. As illustrated, a hat incorporates several sPPE features completely around its brim to generate a protective shield, such as UV energy, aerosols, humidity, heat, ions, and the like that kill or degrade the targeted pathogenic microorganism within the area of effect. This implementation may also be combined with the embodiment of FIG. 6 to doubly protect the user.

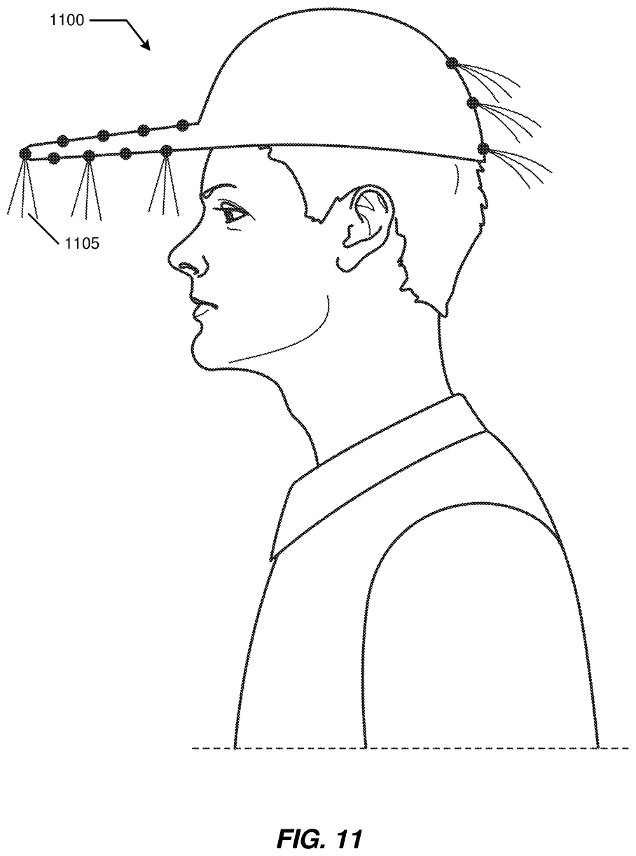

FIG. 11 illustrates an article of head wear 1100 that incorporates another alternative sPPE feature 1105. As illustrated, the sPPE features of FIG. 10 are adapted for use in a cap rather than a hat with a full brim.

“Personalized protective electromagnetic/chemical field” PPE/CF is an alternative term for a personal protective zone. A personalized protective electromagnetic/chemical field PPE/CF may employ far UVC light in the vicinity of (about 207 nm to 222 nm) which may have microbicidal properties for organisms such as H1N1 and influenza, and may sometimes be herein referred to herein as a “personalized UVC field” PUVCF. Multiple PPE/CFs may become spatially localized and produce a “distributed disinfection field” (DDF) as an example of distributed protective zone, where each physically separated PPE/CF operates as a germicidal node or beacon, but together, they collectively produce a global aggregate disinfecting/degrading system, that may be much stronger than an individual PPE/CF. DDF may have certain characteristics: (a) autonomous virucidal/bactericidal/fungicidal beacons, (b) in communication with each other to decrease pathogen count on surface and in air; (c) a system that may tolerate failure; (d) a structure that is dynamic and changes during execution of the distributed function; and (e) may include a distribution over a cluster or cloud having a size/character that may increase, decrease, or change based on number, proximity, and character of individual participating nodes reinforcingly aggregated together, among other factors.

As an enhancement to conventional surgical attire for a surgeon performing a procedure, a personalized DDF with multiple nodes surrounding a single person (e.g., surgeon's body) may be called a “multi-noded PPE/CF”. This is useful for decreasing surgical site infections SSI in the OR space. Everyone in the OR may employ a “multi-noded PPE/CF” to help create a “super distributed disinfection field” SDDF in the space that reduces risks to the patient and to the healthcare team.

The medical and surgical fields have known use of non-personal UVC lamps (often mounted to a ceiling or sidewall) to disinfect surfaces and operative theaters for many years. The UVC light provides a great advantage as noted in this application in that it does not provide a surface or residual upon which a colonization of pathogens can occur.

In spite of its use for over 50 years, there is no clearly defined science or guideline as to effectiveness of UV light based on distance, exposure time and angle of shine. Some studies have suggested that direct central overhead shining of UV light is more effective and efficient in killing pathogens than UV tubes fixed over the side walls. Additionally, some studies have suggested that an effectiveness of the UV light decreases over distance of greater than 7 to 8 feet. In general, the optimal effectiveness with respect to angle of shine, distance and exposure time has not been fully elucidated.

Recent studies have shown that Far-UVC can be utilized as a new tool to control the spread of airborne-medicated microbial diseases such as influenza and tuberculosis. There is a potential that this modality may assist in control of a spread of SARS-CoV-2 “novel coronavirus” which has caused the recent pandemic and associated mortality, morbidity, social and economic devastation.

There has been a distinction between the safety profile of UVC ultraviolet light and Far-UVC ultraviolet light in the vicinity of (207 to 222 nm) range.

This so-called FAR-UVC light cannot penetrate the outer non-living layer of human skin or eye, however, because bacterial and viruses are of micrometer and nanometer dimensions, FAR-UVC light efficiently kills airborne aerosolized viruses and (likely other pathogens including bacteria and fungi) with very low doses, such as a dose sufficient to degrade a pathogen without harming humans and their pets.

It has been shown that far-UVC light at low doses of 2 mJ/cm2 of 222-nm light inactivates >95% of aerosolized H1N1 influenza virus. Extending an OR model, a use continuous very low dose-rate of far-UVC light in indoor and outdoor public locations such as bars, restaurants and businesses can be promising, safe and an inexpensive tool to reduce the spread of airborne- medicated viral and microbial disease. However, as further described herein, UVC lamps mounted to a wall or ceiling may provide some protection, but that protection can be improved by implementations of changes as described herein.

This application includes a description of structures and systems for a personalized protective electromagnetic/chemical field (PPE/CF) that surrounds the body of the wearer of personal protective equipment (PPE), to protect against airborne and surface microbes and pathogens.

Personalized UVC Field (PUVCF) is one embodiment of such personalized protective electromagnetic/chemical field (PPE/CF), which is created by a PPE that is worn and is configured to shine far-UVC light within selected volumes around a face and/or hands of a user.

It is possible that a PUVCF or any “PPE/CF” is significantly more effective and safer than a large and centrally directed light bulb (tube) producing rays of UV light (or any electromagnetic wave) that is shone or beamed from a distance; such as from the ceiling and or the side wall. This type of use may be typically experienced in a hospital, restaurant, sporting event, and/or inside a building by a business entity.

Particularly as ceiling or wall mounted UVC systems have the light pattern independent of where people are located or where they move. Also, this type of protection is not localized to the user so any actual protection will inversely vary as a function of a distance of the user from the wall/ceiling mounted light (sometimes this is referred to as an inverse square law (1/r²) or for some parameters they may fall of more rapidly as (1/r⁴)). For PPE features such as far-UVC, for a given intensity level, may have a lower pathogenic efficacy as to a more complete UVC solution. Therefore, fixed far-UVC generators, such as attachment to ceilings or walls, may be even less effective than a personally-associable/portable user-centric solution.

The inverse square law describes the intensity of light at different distances from a light source. Every light source is different, but the intensity changes in the same way. The intensity of light is inversely proportional to the square of the distance. This means that as the distance r from a light source increases, the intensity of light is equal to a value multiplied by 1/r². Many sPPE features may obey an inverse power law, which may further enable communal protection in overlapping zones. Further, this communal protection is stronger the closer users are to each other.

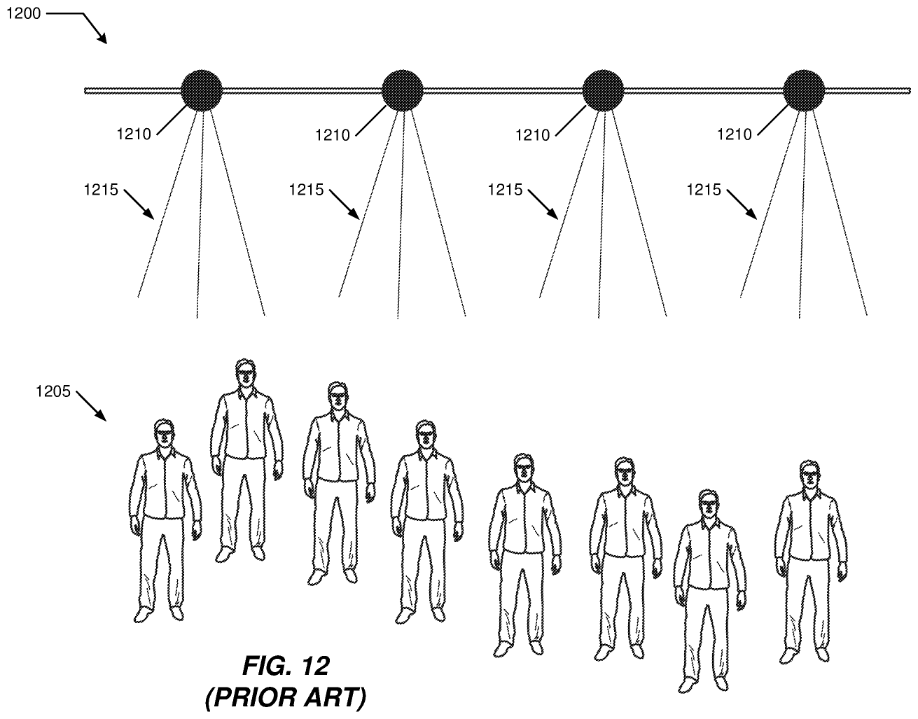

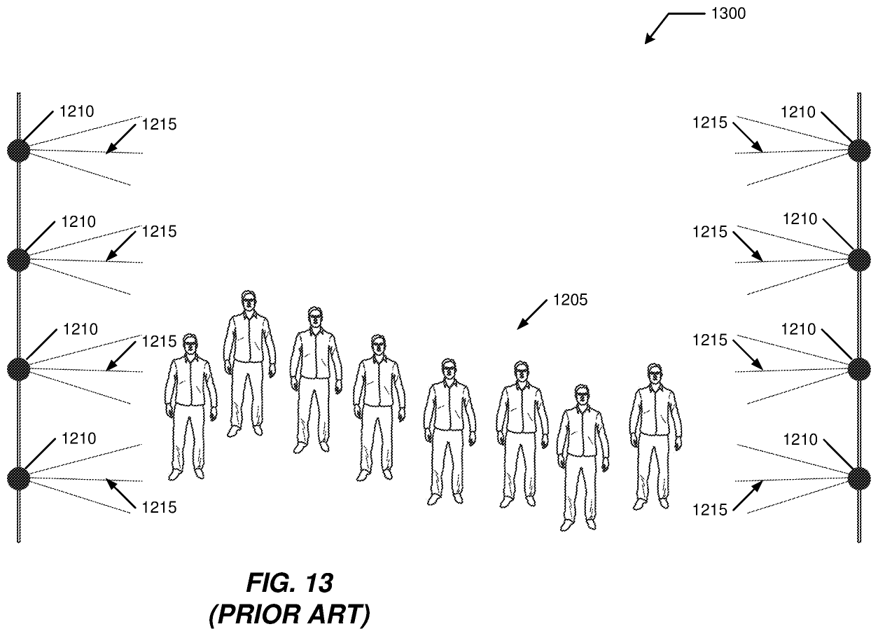

FIG. 12 illustrates a conventional ceiling-mounted UV distribution system 1200 and FIG. 13 illustrates a conventional wall-mounted UV distribution system 1300. Each system includes a collection of people 1205 within an interior space, people 1205 are desirably protected from airborne pathogens (for purposes of this discussion without physical barriers such as masks, shields, gloves, and/or gowns).

Each system further includes a set of mounted UVC lamps 1210 (system 1200 mounts these to the ceiling and system 1300 mounts these to side walls) that shine UVC light 1215 into the interior space. System 1200 and system 1300 bathe the interior space with UVC that obeys the inverse square law with respect to each source. Thus, the quality of protection may vary, and vary significantly, based upon location and there is no inherent response of the level of protection based upon location of individual members of the group or other environmental characteristics and occupancy.

In contrast to UVC lamps mounted to a ceiling (FIG. 12) or a wall (FIG. 13) a combination (or plurality) of PUVCFs may provide a significantly more enhanced and logarithmic increase in microbicidal (viricidal, bactericidal, and/or fungicidal) activity. This new concept considers the possibility that the higher the number of individuals present in an interior space (such as a place of business, gym, store, salon, and the like), the more protection (microbicidal activity) is provided by the plurality of PPE/CFs—referred to herein sometimes as herd protection. This herd protection may be available for many different types of non-physical barrier microbicidal agents/agencies that may provide a reinforcing personal protective zone as one protection zone of one user begins to overlap with another protection zone of another user. Herd protection may be a consequence of some implementations of the distributed disinfection fields (DDF).

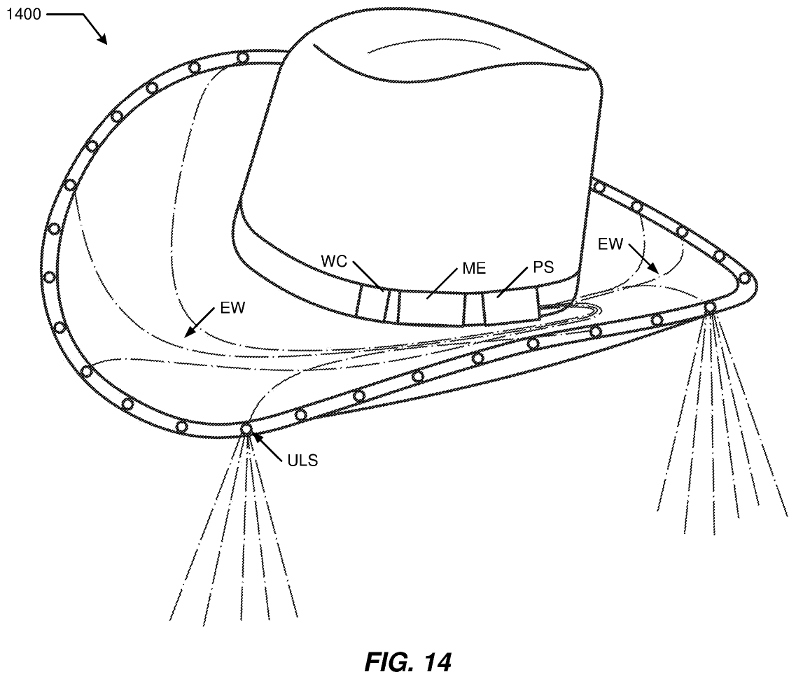

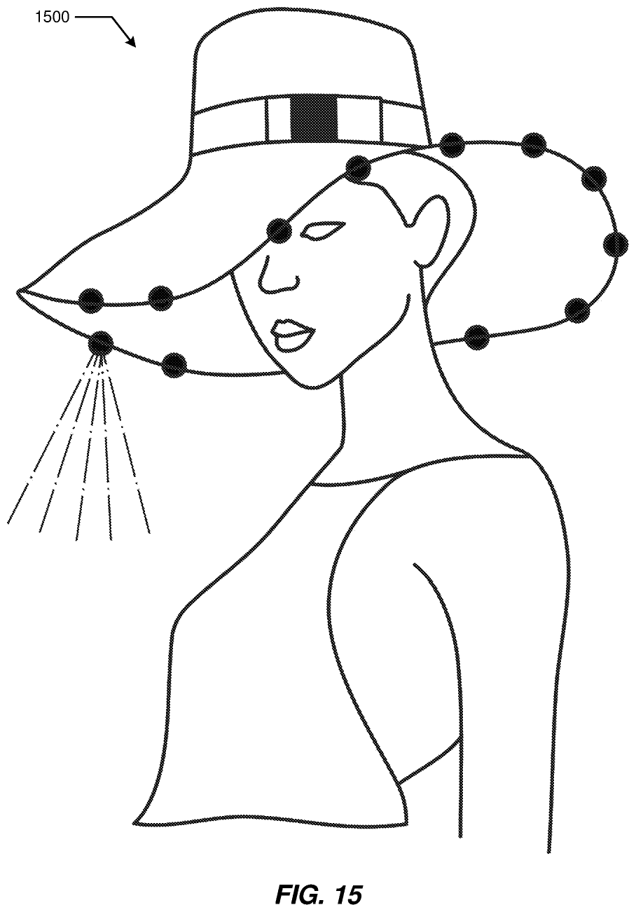

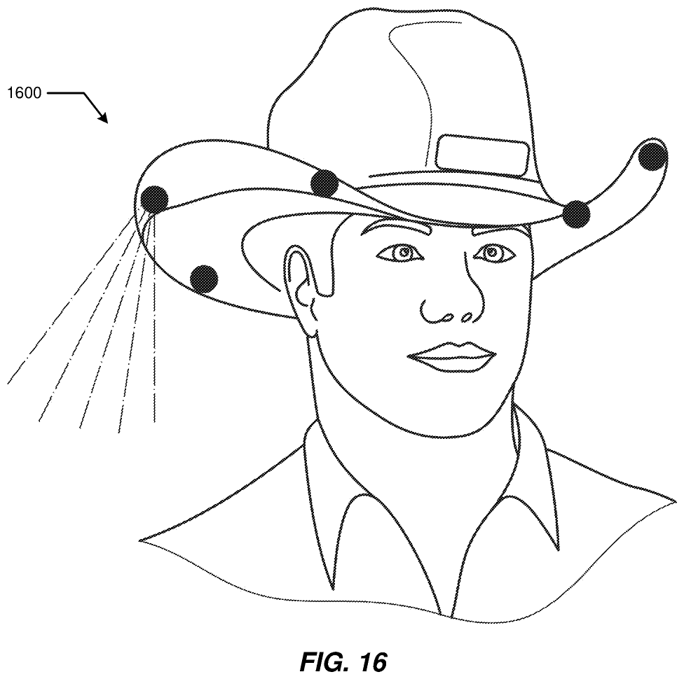

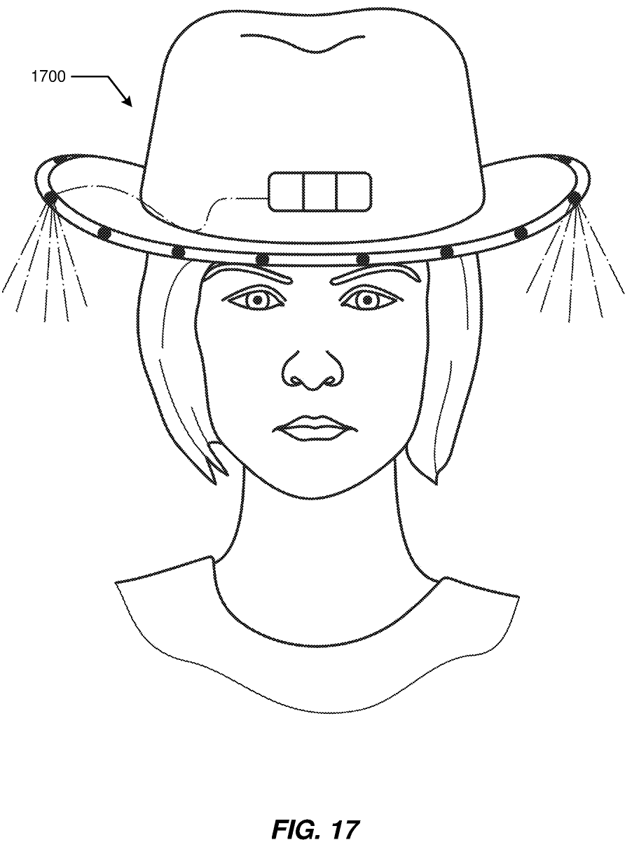

FIG. 14-FIG. 17 depict systems incorporating a PPE/CF (as an example PUVCF) in a variety of fashionable wearable equipment that could be considered socially unencumbered accessories. FIG. 14 illustrates a first hat 1400 with an architecture providing PUVCF; FIG. 15 illustrates a second hat 1500 with an architecture providing PUVCF similar to FIG. 14; FIG. 16 illustrates a third hat 1600 with an architecture providing PUVCF similar to FIG. 14; and FIG. 17 illustrates a fourth hat 1700 with an architecture providing PUVCF similar to FIG. 14.

These hats may include (a) a power supply (PS), (b) a set of miniaturized electronics (ME) including a microprocessor executing a set of instructions retrieved from a memory (e.g., a stored program computing platform), (c) an electronic wiring-circuitry (EW), (d) wireless communications (WC), and (e) far-UVC light sources (ULS). Some implementations may include configuration/deployment of distributed disinfection fields (DDF) for protection against spread of airborne mediated microbial, viral, and fungal disease.

As noted use of UV light in the medical field has so far been centralized where large germicidal UV tubes such as the 40-Watt power Phillips Holland Lamp are hung from the wall or ceiling at a certain distance (i.e. 8 feet) for a certain amount of time (i.e. 30 minutes) to inactivate pathogens.

The distributed operating field where physically separated “PPE/CFs” operate as “germicidal nodes” or “germicidal beacons”. These distributed nodes may operate at much lower power (wattage) that is much less harmful and therefore safer to the human body; while a field protection intensity may be greater at internodal locations between users.

For example, each individual node or beacon of UVC light works as a subset of a global aggregate operating system. Each beacon of light may interact with another beacon and with a central system to achieve a common goal of safely inactivating and killing microbes, bacteria and viruses on surfaces and within the air; or at a minimum reduce the viable count of pathogens in air and on surfaces.

The concept of distributed fields for disinfection (DDF) is described in contradistinction to the centralized system that is currently used to disinfect.

The properties of a distributed disinfection Field DDF may include one or many of the following: (i) there are several autonomous beacons (nodes) independently producing microbicidal properties, (ii) the beacons communicate with each other to solve a common problem, that is to decrease the viable count of pathogens in air and on surfaces, (iii) the system tolerates failure in individual beacons as some users may be included with a group protection zone produced from the reinforcing/overlapping individual protection zones, (iv) the structure of the system may not be known in advance and may change during the execution of the distributed function as it may be an ad hoc response to the deployment of many reinforcing/overlapping personal protection zones within an enclosed space, (v) disinfection is distributed across a cluster, grid or cloud, and may be enhanced as the number of beacons (nodes) are increased.

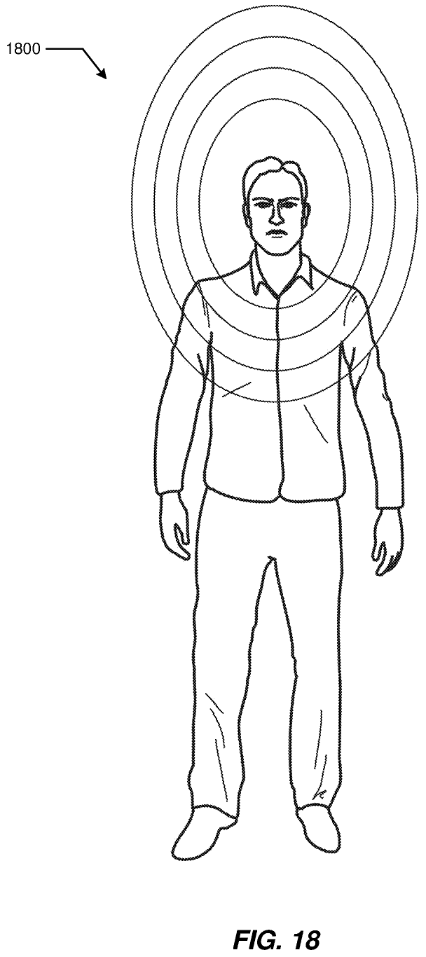

FIG. 18 illustrates a representation of a person 1800 equipped with a personal wearable protective-zone generation system. The actual generation system may be any of a variety of protective field projector as described or suggested herein and the actual provision of the personal protection zone may vary greatly from that depicted in FIG. 18, including location, shape, and zone properties/composition.

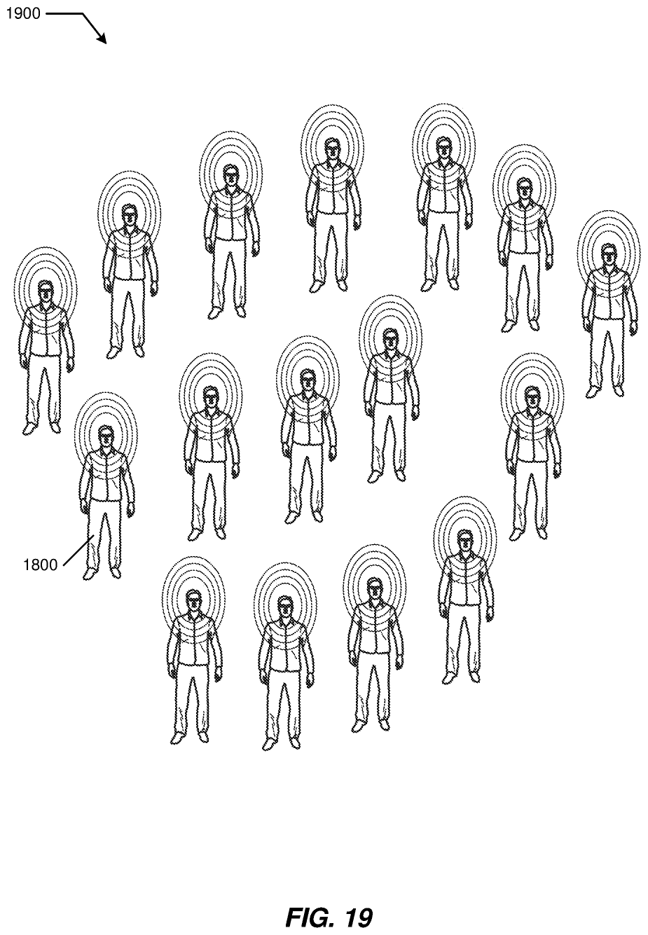

FIG. 19 illustrates a first gathering 1900 of a first set of persons 1800, each person 1800 equipped with a protection zone projector, the gathering 1900, maybe more precisely the collection of reinforcing protection zone projectors collectively producing a group protective zone when the personal protection zones include characteristics of being able to be synergistically combined by reinforcing overlapping of personal protection zones (for example, an intensity of the group protection zone in an overlapping region has a greater microbicidal activity than a microbicidal activity contribution from each individual personal protection zone in the overlapping region.

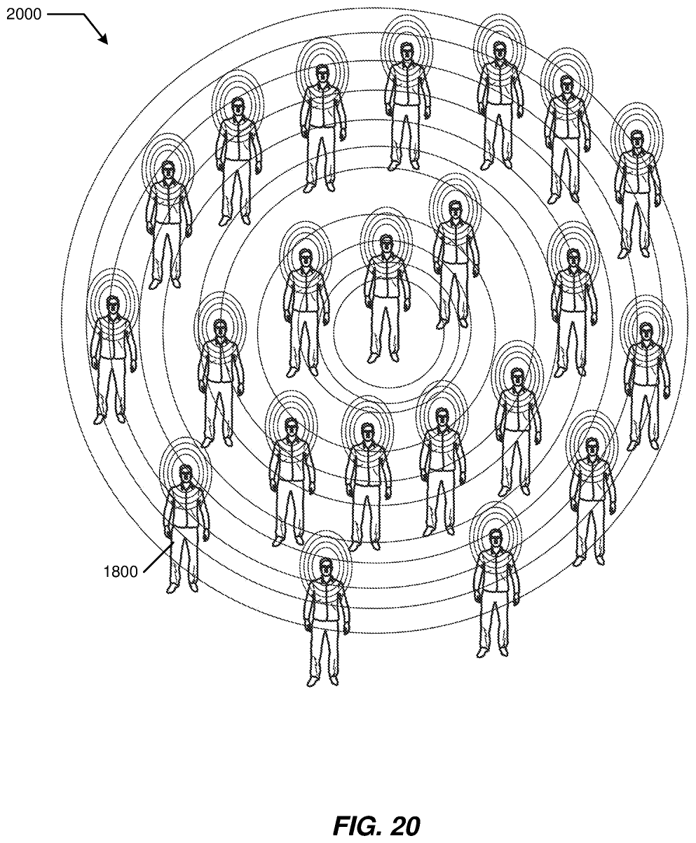

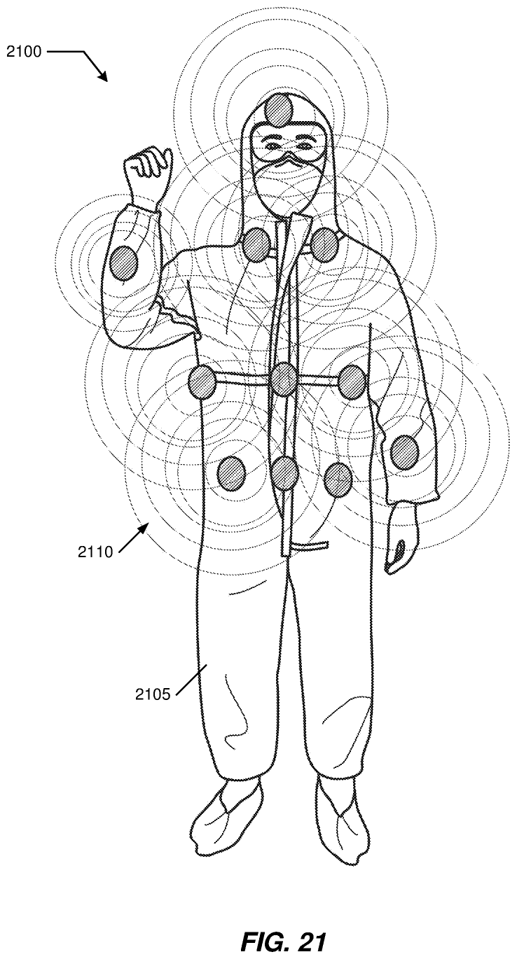

FIG. 20 illustrates a second gathering 2000 of a second set of persons 1800 depicting an increased level of protective intensity, directly related to the number and proximity for the distributed reinforcing group protective zone illustrated in FIG. 21.

FIG. 21 illustrates a personal protective equipment 2100 including a combination of conventional technology 2105 and field projectors 2110 as described herein to improve protection for first responders and health care professionals frequently in close proximity to third-parties who are infected or whose infection status is unknown - the concept of DDF may be applicable to the surgical field or other environments where there are potentially airborne pathogenic microorganisms and/or a desire to reduce a presence of potentially pathogenic microorganisms that may exist in the environment.

The cost of surgical site infections (SSI) to society is huge. SSIs are the most common and costly of all hospital-acquired infections, accounting for 20 percent of all hospital- acquired infections. They occur in an estimated 2 percent to 5 percent of patients undergoing inpatient surgery. The estimated annual incidence of SSIs in the U.S. ranges from 160,000 to 300,000, and the estimated annual cost ranges from $3.5 billion to $10 billion. On average, a surgical site infection increases the hospital length of stay by 9.7 days, according to studies cited in the guidelines.

In truth there has been no innovation in surgical gown and glove technology over the last century. Surgical gown and gloves became common place in the late 19th and early 20th century.

Some implementations may include combined concepts of PPE/CF and DDS for the surgeon's gown, face shield/mask/helmet, and glove. An embodiment would involve surgical gown, mask and gloves with multiple nodes producing a distributed cooperative nodes of PUVCF surrounding the surgeon's body.

In certain situations, the operating room staff including the surgeon, the scrubbed assistant and the circulating nurses can all be wearing specialized gowns with multi-noded PPE/CFs; which then can produce a DDF not only for the individual but for the entire operating room space. Currently, there is a significant effort to minimize staff movement within the operating room, in order to minimize the chance of SSI, however, the traffic in the OR is usually constant.

When all operating room/activity space staff wear some version of [DDF+PPE/CF] gowns, it is conceivable that a super distributed disinfection field (SDDF) is created within the operating room/activity space, with the potential to significantly reduce the chance of surgical site infections (SSI).

Embodiments and implementations may include one or more features described above characterized/enhanced/modified as follows. Protection zone projectors need not be homogenous—that is a particular solution may include a set of projectors with some projectors of that set providing one microbicidal solution and some projectors of that set providing another different microbicidal solution.

Further, the various projectors may not always operate together and at the same intensity when they do operate together. For example, in some instances projectors around a perimeter of a hat brim may operate in different regions—a forward region and a rearward region, or those two plus left hand side and right hand side regions, among many other different permutations for directionality of region-assigned projectors. Different regions may be selectively active at any given time based upon user choice/environmental conditions.

For variable intensity projectors, such as those in different regions, it may be desirable to have forward region set of projectors at a higher intensity than rearward region set of projectors (e.g., when the user is running along a trail—and in some cases, a faster that the user runs, the greater intensity of the forward region component). It may be desirable to have forward/region projectors at a reduced intensity as compared to left-hand/right-hand side region projectors (e.g., when the user is seated on a plane, train, bus, or other transit vehicle having persons closer on the sides than in the front/back). Some implementations may include a broad spectrum “background” level of protective agents at all time, and when remote from other people, the power level may be set at a low level.

Such solutions may include proximity detectors to determine when other people get close, such as within a distance when protective microbicidal protocols must or should be used, and/or directional sensors to determine a proximity/number of people in different regions and set intensity/agency type accordingly. (Such systems may, for example, alert a user and approaching people when they approach within a predetermined distance, say about six feet (or other social distancing separation distance). The alert may remind the approaching people to put on/adjust their protective solution and start the personal protection generators (as appropriate) in an event they are not configured to react to the person's approach.

Some systems may include communications equipment that allow one personal protective zone solution from one user to negotiate with another personal protective zone solution from another user to determine both the parameters and characteristics of each personal protection zone, but also the parameters and characteristics of the ad hoc group protection zone defined by their overlapping personal protection zones. One user may provide microbicidal agents A and B and another user may provide microbicidal agents A and C—with the group protection zone including agents A, B, and C. The negotiation may implement different solutions—such as maintaining the intensities of the individual protective zone projectors possibly producing an increased intensity in the overlapping regions. In other solutions, each projector may reduce its power so that the intensity of the overlapping regions meets a desired threshold intensity, which may also help conserve power.

These systems may include a general stored program computer with memory (e.g., storing instructions and data) and a microprocessor responsive to the instructions to control the personal projectors. This control may be based upon selection of an appropriate predetermined one configuration for intensities/composition of projectors from a set of configurations. For example, a configuration for inside dining with fewer than 10 people in the room or a configuration to participation in a night club, concert, or travel on various public transit solutions. The user may choose the most applicable configuration and the personal protection generators will implement the desired preconfiguration. In other situations, configuration information for projector settings appropriate to each venue may be provided to the systems from a third party (such as the venue operator), which implement that third-party configuration.

Conditions in a venue may change over time, such as from an opening of a nightclub to max occupancy and activity of users within the nightclub. As long as each person is equipped with a personal projector system then the users have measure of protection. When a sufficient number of these personal projectors are responsive to variable configuration information, then the measure of protection may be improved, particularly when the correct configuration is available and active. When the venue conditions affecting projector configurations are measured/sensed (by the individual user system and/or from onsite venue data collectors) are updated appropriately as venue conditions change, the users and patrons of the venue may receive an enhanced level of protection.

In other words, for some implementations smart features in a venue and incorporated into personal protective zone solutions allow the entities to negotiate the best settings, in some cases in realtime/near realtime, for creating the reinforcing protective fields tuned for the particular possible pathogenic threats that may be most likely to exist in the venue at any given time.

When entering into a venue, operational parameters could be loaded into the various personal controllers. Similarly, when different jurisdictions (cities, states, countries) want to change/adapt the protective zone configurations, that could be easily established by a variable intensity non-homogenous set of multiple protective zone projectors.

As an understanding of various risks and a distribution of those risks are known or as the knowledge develops, it may be desirable to have different projectors more effective than others at different threats, and then adapt multimode ppe emitters accordingly based upon risk profiles rather than actual sensor telemetry. Each jurisdiction may compile and publish these risks so users may respond accordingly. For example, as a number of confirmed cases of a particular pathogen increase in geographic locale, users in that locale may tune their systems against that particular pathogen. When the number of confirmed cases is high, the user may walk around with a relative intensity set of 8 out of 10. However, when the number of cases is low, the user may configure their system at a relative intensity of 2 out of 10 (10 max).

As noted above, for venues with super spreaders or super spreading events, it may be desired to require all patrons to set their individual solutions at or near maximum.

Some solutions may be power hungry, especially in solutions disposed in small/discreet form factors. It is known to use wireless power solutions in which power transmitters may transmit operating power to a remote system. In lieu of, or in addition to the ceiling/wall mounted UVC emitters, a venue may include power transmitters to power these solutions. The venue is thus enhancing the safety of its patrons. Control and effective pathogen transmission control allows the various applicable health departments to permit the venue to continue to operate, thus there is a mutual interest in the venue operator and the users in efficient long-term control of pathogens.

A microorganism, including viruses, can become airborne. Contaminated material can be aerosolized in many different ways, ranging from wind to human and animal activities such as sneezing, mechanical processes, and the like. When the aerodynamic size of an infectious particle is appropriate, it can remain airborne, come into contact with humans or animals, and potentially cause an infection. The probability of an airborne microorganism-laden particle causing an infection depends on its infectious potential and its ability to resist stresses associated with aerosolization.

Airborne microorganisms can represent major health and economic risks to human and animal populations. Appropriate preventive actions can be taken when threats posed by such microorganisms is better understood. People need to be aware of the nature, concentration, and pathogenicity of airborne microorganisms to better control them. This information can be obtained by using various air sampling methods, each of which has its particular advantages and disadvantages. Many types of samplers and analytical methods have been used over the years. A system or method incorporating the present invention may incorporate an ambient sensing apparatus to determine/assess user risk from ambient airborne microorganisms and may adjust PPE features in response to such determinations/assessments. In some implementations, a third-party such as a venue operator (e.g., operators of a tavern, bar, nightclub, gym, salon, tattoo parlor, movie theatre, sports stadium, transit vehicle, religious service, dining/restaurant, and other businesses having gatherings of multiple persons within confined spaces. An interface communicated to the controller may receive a quantitative or qualitative ambient microorganism characterization. The characterization may be received by the interface from the user (e.g., manually entered), from an onboard system or a third-party providing environmental data wirelessly (automatic), or combinations thereof. For example, the microelectronics (ME) system illustrated in FIG. 14 may incorporate a sensing structure to collect ambient environment information about airborne microorganisms and other systems to evaluate such microorganisms to assist the system in determining the nature of the user's personal protective zone (or distributed zone(s) when collaborating with the systems of other users in the same ambient environment).

A feature of the present invention allows for implementations that may offer broad spectrum PPE features that are designed to kill/degrade a wide variety of “common” pathogenic microorganisms, including prokaryotes, eukaryotes, and viruses having RNA or DNA. Systems such as this may be appropriate to wear at all times, even when not in a pandemic situation. In some cases, a targeted PPE feature may be desired in which the killing/degrading modality of any feature is tuned to a specific pathogenic microorganism, such as a novel corona virus. That is a particular temperature or UV wavelength may be optimally effective at killing/degrading and the PPE feature(s) may be adjusted/configured accordingly. In some embodiments, the wearable may be tuned/adjusted by the user, such as for particular risk profiles. The Internet of Things (IOT) includes features that may allow some potential geolocation features to access and set/tune a user's PPE features to automatically deploy appropriate PPE feature settings and conditions based upon location or other metric (and in some cases turning them ON/OFF) based upon approved safety guidelines.

Some embodiments of the present invention may also be adapted for other environmental hazards in addition to pathogenic microorganisms. Air flow/pressure differential systems may also address ambient particulates disposed in the air by physically moving the particles away using tailored airflow PPE features. Further, some non-microorganism particulate remediation may be achieved by using electrostatic PPE features when undesired ambient particulates include a net electrical charge, either naturally or altered by the PPE feature.

The system and methods above have been described in general terms as an aid to understanding details of preferred embodiments of the present invention. In the description herein, numerous specific details are provided, such as examples of components and/or methods, to provide a thorough understanding of embodiments of the present invention. Some features and benefits of the present invention are realized in such modes and are not required in every case. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

It will also be appreciated that one or more of the elements depicted in the drawings/figures can also be implemented in a more separated or integrated manner, or even removed or rendered as inoperable in certain cases, as is useful in accordance with a particular application.

Additionally, any signal arrows in the drawings/Figures should be considered only as exemplary, and not limiting, unless otherwise specifically noted. Combinations of components or steps will also be considered as being noted, where terminology is foreseen as rendering the ability to separate or combine is unclear.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims. Thus, the scope of the invention is to be determined solely by the appended claims.

What is claimed as new and desired to be protected by Letters Patent of the United States is:

1. A system for a user, the system providing individual protection against a pathogenic microorganism, comprising:
a set of components configured for association with the user, said set of components adapted to move with the user during operation, said set of components including a source of power, a controller, coupled to said source of power, executing a set of instructions retrieved from a memory to produce a set of control signals, and a set of microbicidal generators, coupled to said source of power and responsive to said set of control signals, providing a structureless microbicidal agency projected within a protective zone proximate a set of anatomical features of the user, wherein said microbicidal agency destroys, disturbs, denatures, interferes, or otherwise reduces a pathogenicity or transmission of a pathogenic microorganism within said protective zone before said pathogenic microorganism contacts one said anatomical feature of said set of anatomical features.

2. The system of claim 1 wherein said set of anatomical features includes one or more features selected from the group consisting of eyes, nose, mouth, face, hands, fingers, skin, hair, mucosa, and combinations thereof.

3. The system of claim 1 wherein said microbicidal agency includes one or more sPPE features selected from the group consisting of heat, drying/desiccation function, UV light including far-UVC light, ozone 03, ions, ions mixed with peroxide, visible light, visible pulsed light, humidity greater than ambient, ionizing or non-ionizing radiation, with energy configured for sufficient degradation of a targeted pathogenic microorganism(s) while having an energy level within a safe threshold range for the user, an aerosol of microbicidal agent(s), pulsed laser tuned to a degrading/destructive resonance frequency of a class of pathogenic microorganisms including said targeted pathogenic microorganism or a specific targeted pathogenic microorganism, ultrasonic energy which may also be tuned to a degrading resonance frequency, air pressure phenomena (negative and positive air pressures moving air proximate said set of anatomical features), microbicidal agents, microbiostatic agents, and combinations thereof.

4. The system of claim 1 wherein said microbiocidal agency includes a microbiocidal field having a pathogenic efficacy characterized by an inverse power law.

5. The system of claim 1 wherein the pathogenic microorganism includes a bacterium, a virus, a viroid, a fungus, an archaea, and combinations thereof.

6. The system of claim 5 wherein said virus includes a corona virus.

7. The system of claim 6 wherein said corona virus includes SARS-CoV-2 or other virus or microbe contributing to COVID-19.

8. The system of claim 1 wherein said set of components are incorporated into a set of wearables for the user.

9. The system of claim 8 wherein said set of wearables includes one or more articles selected from the group consisting of clothing, jewelry, fashion accessories, masks, gowns, helmets, face shields, hazmat suits, surgical gowns, respirators, and combinations thereof.

10. The system of claim 1 further comprising an interface receiving environmental data and wherein said controller, responsive to said environmental data, configures said set of microbicidal generators.

11. The system of claim 10 where said environmental data includes particular microbiocidal parameters relating to an ambient pathogenic microorganism present in an ambient environment of the user, said particular microbiocidal parameters configured to tune said set of microbiocidal generators for attacking specifically said ambient pathogenic microorganism.

12. The system of claim 1 wherein the user includes a first user, wherein said set of components includes a first set of components having a first controller providing a first set of control signals, wherein said set of microbiocidal generators includes a first set of microbiocidal generators responsive to said first set of control signals, wherein said protective zone includes a first protective zone for said first user, further comprising:
a second set of components configured for association with a second user different from said first user, said second set of components adapted to move with said second user during operation, said second set of components including a source of power, a second controller, coupled to said source of power, executing a set of instructions retrieved from a memory to produce a second set of control signals, and a second set of microbicidal generators, coupled to said source of power and responsive to said second set of control signals, providing a second microbicidal agency projected proximate a set of anatomical features of said second user, wherein said second microbicidal agency destroys, disturbs, denatures, or otherwise reduces a pathogenicity or transmission of a pathogenic microorganism within a second protective zone for said second user before said pathogenic microorganism contacts said set of anatomical features of said second user.

13. The system of claim 12 wherein said users are spaced apart a distance D, wherein said protective zones overlap at locations intermediate pair sets of said users producing an overlapping protective zone including contributions from both said first protective zone and said second protective zone, and wherein a microbicidal activity of said overlapping protective zone is greater than any contribution from any contributing protective zone from any single user of said pair sets.

14. The system of claim 13 wherein said controllers include a set of communications components configured to communicate with other controllers and exchange communal information, said controllers responsive to said exchanged communal information to configure collectively said overlapping protective zone through configuration of individual protective zones provided to each user.

15. The system of claim 1 wherein said controller includes a power communication system wherein said power source receives wirelessly power from a remote power source operating within an ambient environment of the user.

16. The system of claim 1 further comprising an ambient sensing system configured to evaluate an ambient environment for an ambient pathogenic microorganism, said ambient sensing system coupled to said controller wherein said controller reconfigures said control signals for a sensed ambient pathogenic microorganism.

17. The system of claim 1 wherein said set of components define an sPPE and further comprising a conventional personal protective equipment solution coupled to said sPPE providing a hybrid PPE combining cPPE and sPPE protective elements.

18. The system of claim 17 wherein said cPPE includes surgical personal protective equipment worn in an operating venue.

19. A method providing individual protection to a user against a pathogenic microorganism, comprising:
associating a set of sPPE components with the user;
moving said set of components in synchronicity with a movement of the user during operation of said set of sPPE components; wherein said set of sPPE components include a source of power, a controller, coupled to said source of power, executing a set of instructions retrieved from a memory to produce a set of control signals, and a set of microbicidal generators, coupled to said source of power and responsive to said set of control signals; and
projecting a structureless microbicidal agency within a protective zone proximate a set of anatomical features of the user, wherein said microbicidal agency degrades, destroys, disturbs, denatures, deactivates, interferes, or otherwise reduces a pathogenicity or transmission of a pathogenic microorganism within said protective zone before said pathogenic microorganism contacts one said anatomical feature of said set of anatomical features.

20. The method of claim 19 wherein said set of anatomical features includes one or more features selected from the group consisting of eyes, nose, mouth, face, hands, fingers, skin, hair, mucosa, and combinations thereof.

21. The method of claim 19 wherein said microbicidal agency includes one or more sPPE features selected from the group consisting of heat, drying/desiccation function, UV light including far-UVC light, ozone 03, ions, ions mixed with peroxide), visible light, visible pulsed light, humidity greater than ambient, ionizing or non-ionizing radiation, with energy configured for sufficient degradation of a targeted pathogenic microorganism(s) while having an energy level within a safe threshold range for the user, an aerosol of microbicidal agent(s), pulsed laser tuned to a degrading/destructive resonance frequency of a class of pathogenic microorganisms including said targeted pathogenic microorganism or a specific targeted pathogenic microorganism, ultrasonic energy which may also be tuned to a degrading resonance frequency, air pressure phenomena (negative and positive air pressures moving air proximate said set of anatomical features), microbicidal agents, microbiostatic agents, and combinations thereof.

22. The method of claim 19 wherein said microbiocidal agency includes a microbiocidal field having a pathogenic efficacy characterized by an inverse power law.

23. The method of claim 19 wherein the pathogenic microorganism includes a bacterium, a virus, a viroid, a fungus, an archaea, and combinations thereof.

24. The method of claim 23 wherein said virus includes a corona virus.

25. The method of claim 24 wherein said corona virus includes SARS-CoV-2 or other virus or microbe contributing to COVID-19.

26. The method of claim 19 wherein said set of components are incorporated into a set of wearables for the user.

27. The method of claim 26 wherein said set of wearables includes one or more articles selected from the group consisting of clothing, jewelry, fashion accessories, masks, gowns, helmets, face shields, hazmat suits, surgical gowns, respirators, and combinations thereof.

28. The method of claim 19 further comprising receiving environmental data through an interface and wherein said controller, responsive to said environmental data, configures said set of microbicidal generators.

29. The method of claim 28 where said environmental data includes particular microbiocidal parameters relating to an ambient pathogenic microorganism present in an ambient environment of the user, said particular microbiocidal parameters configured to tune said set of microbiocidal generators for attacking specifically said ambient pathogenic microorganism.

30. The method of claim 19 wherein the user includes a first user, wherein said set of components includes a first set of components having a first controller providing a first set of control signals, wherein said set of microbiocidal generators includes a first set of microbiocidal generators responsive to said first set of control signals, wherein said protective zone includes a first protective zone for said first user, further comprising:
a second set of components configured for association with a second user different from said first user, said second set of components adapted to move with said second user during operation, said second set of components including a source of power, a second controller, coupled to said source of power, executing a set of instructions retrieved from a memory to produce a second set of control signals, and a second set of microbicidal generators, coupled to said source of power and responsive to said second set of control signals, providing a second microbicidal agency projected proximate a set of anatomical features of said second user, wherein said second microbicidal agency destroys, destroys, disturbs, denatures, or otherwise reduces a pathogenicity or transmission of a pathogenic microorganism within a second protective zone for said second user before said pathogenic microorganism contacts said set of anatomical features of said second user.

31. The method of claim 30 wherein said users are spaced apart a distance D, wherein said protective zones overlap at locations intermediate pair sets of said users producing an overlapping protective zone including contributions from both said first protective zone and said second protective zone, and wherein a pathogenicity of said overlapping protective zone is greater than any contribution from any contributing protective zone from any single user of said pair sets.

32. The method of claim 31 wherein said controllers include a set of communications components configured to communicate with other controllers and exchange communal information, said controllers responsive to said exchanged communal information to configure collectively said overlapping protective zone through configuration of individual protective zones provided to each user.

33. The method of claim 19 wherein said controller includes a power communication system wherein said power source receives wirelessly power from a remote power source operating within an ambient environment of the user.

34. The method of claim 19 further comprising an ambient sensing system configured to evaluate an ambient environment for an ambient pathogenic microorganism, said ambient sensing system coupled to said controller wherein said controller reconfigures said control signals for a sensed ambient pathogenic microorganism.

35. The method of claim 19 wherein said set of components define an sPPE and further comprising a conventional personal protective equipment solution coupled to said sPPE providing a hybrid PPE combining cPPE and sPPE protective elements.

36. The method of claim 35 wherein said cPPE includes surgical personal protective equipment worn in an operating venue.
